(19) United States
(12) Patent Application Publication
(10) Pub. No.: US 2009/0004306 A1
(43) Pub. Date: Jan. 1, 2009

(54) Composition for treatment of diabetes and maintaining healthy blood glucose levels

(76) Inventors: Heather Renee Pridemore, Madison, TN (US); Mark Carroll Faulkner, Nashville, TN (US); Mohammad A.R. Jawad, London (GB)

Correspondence Address: VIREO SYSTEMS , INC, 305 WILLIAMS AVENUE, MADSON, TN, 37115, US

(21) Appl. No.: 11/768,405

(22) Filed: Jun. 26, 2007

(51) Int. Cl. A61K 36/00; A61K 36/48; A61K 36/54; A61P 3/10

(52) U.S. Cl. 424/739; 424/725

(57) ABSTRACT

A medicinal composition is presented for the treatment of diabetes in human patients. The medicinal composition of the invention includes hypoglycemic properties that allows some patients to discontinue use of anti-diabetic pharmaceuticals in favor of this medicinal composition. The medicinal composition of the invention allows human patients to maintain healthy blood glucose levels.

### REFERENCES CITED

Anderson 2000

Gaby 1996

Kim et al 1995

Madar and Stark 2002

McCary 2005

Newairy et al 2002

Raghuram et al 1994

Sharma et al 1990

### DESCRIPTION

### Background of the Invention

1. Field of the Invention

The present invention relates to a medicinal composition useful in the treatment of diabetes in a human patient and a method of treatment of diabetes in a human patient.

The medicinal composition is also useful in maintaining healthy blood glucose levels in a healthy human patient.

2. Description of the Prior Art

It is well known that diabetes is a serious medical condition that develops in a significant number of humans. Diabetes is quite debilitating unless treated. It often leads to complications in other medical conditions a patient may have. Diabetes and its complications can lead to death.

Diabetes is characterized by the inability of the pancreas gland to secrete insulin of a reduced sensitivity to insulin. The role of insulin in the human body is to help the body burn sugar in the cells of the body in order to provide energy. Sugar is not properly metabolized in a diabetic patient, which results in an elevated level of sugar in the blood.

Current methods of controlling glucose metabolism and response are pharmaceutical oral hypoglycemics, diet modification, and insulin. Each of these methods has limitations and can be ineffective in certain individuals. Many nutraceutical and herbal products have been in use for a long time by certain communities as traditional anti-diabetic therapies with proven effectiveness. However, there is still a need for a treatment that will allow humans to get and maintain a healthy blood glucose level.

In human investigations, it has been shown that fenugreek seeds powder promotes the increase in the number of insulin receptor on cells thus making cells more sensitive to lower insulin concentrations (Madar and Stark 2002). Preliminary and double-blind trials found that fenugreek helps improve blood sugar control in patients with insulin-dependent (type 1) and non-insulin-dependent (type 2) diabetes (Sharma et al 1990; Raghuram et al 1994).

The active ingredient in cinnamon was defined as cinnamaledhyde. It was found to exhibit hypoglycemic properties. The results of the Kim study suggest that cinnamon extract has a regulatory role in blood glucose level, and it may also exert a blood glucose-suppressing effect by improving insulin sensitivity or slowing absorption of carbohydrates in the small intestine (Kim et al 2005).

In traditional medicine in many countries, especially in the Middle East, termis is used for its anti-diabetic property. In an experimental study, it has been reported that the hypoglycemic capability of termis could be in part due to the induction of certain liver enzymes. It has been found that the elevated glucose level was restored to its normal level after treatment with termis (Newairy et al 2002).

Ample intakes of chromium picolinate appear to promote insulin sensitivity in many individuals and improve glycemic control in some diabetics (McCarty 2005). In recent years, chromium has been shown to improve glucose levels and related variables in people with glucose intolerance and type 1, type 2, gestational, and steroid-induced diabetes (Anderson 2000; Gaby 1996).

### DESCRIPTION OF THE INVENTION

It has been discovered according to the present invention that a combination of plant products and minerals is an effective treatment of diabetes. This combination of plant products and minerals is also effective in maintaining healthy blood glucose levels. The products possess hypoglycemic activity (i.e. these products have the ability to decrease the level of sugar in the blood). It is possible for some patients to discontinue use of anti-diabetic pharmaceuticals in favor of this medicinal composition.

The medicinal composition for treating diabetes and maintaining a healthy blood glucose level includes fenugreek, cinnamon, termis, and chromium picolinate. All components are mixed together to form an acceptable medium for administering a dose between one and three times per day. If a dose is administered three times per day, the dose will be taken before meals. The amount of the composition administered as a dose is an amount sufficient to result in healthy blood glucose levels for the patient.

The invention has been described with reference to the preferred embodiment. Obvious modifications and alterations may occur to others upon reading and understand the detailed description. It is intended that the invention includes all such modifications and alterations insofar as they come within the scope of the claims or the equivalents thereof.

We claim:

1. A medicinal composition comprising an effective amount of fenugreek, cinnamon, and termis.

2. A medicinal composition according to claim 1 further comprising an effective amount of chromium picolinate.

3. A method of treating diabetes in a human patient comprising administering to said patient the medicinal composition of claims 1 to 2 at least once a day.

4. A method according to claim 3 further comprising administering said medicinal composition three times over a twenty-four hour period.

5. A method of promoting and maintaining healthy blood glucose levels in a human patient comprising administering to said patient the medicinal composition of claims 1 to 2 at least once a day.

6. A method according to claim 5 further comprising administering said medicinal composition three times over a twenty-four hour period.
